(19) United States
(12) Patent Application Publication
(10) Pub. No.: US 2007/0224173 A1
(43) Pub. Date: Sep. 27, 2007

(54) Nonexpansion Protocol for Autologous Cell-Based Therapies

(76) Inventors: Edouard A. Koullick, Golden Valley, MN (US); Tania Marie Schroeder, South St. Paul, MN (US)

Correspondence Address: AMS RESEARCH CORPORATION, 10700 BREN ROAD WEST, MINNETONKA, MN, 55343, US

(21) Appl. No.: 11/567,022

(22) Filed: Dec. 5, 2006

Related U.S. Application Data
(60) Provisional application No. 60/784,305, filed on Mar. 21, 2006.

(51) Int. Cl. A61K 35/32; A61K 38/18; A61K 31/737; A61K 31/728; A61K 31/727; A61K 9/14

(52) U.S. Cl. 424/93.7; 514/12; 514/56; 514/54; 424/486

(57) ABSTRACT

The present application describes various applications of the non-expansion protocol for the preparation of an injectable autologous cell mixture of the present invention that can be used to prevent symptoms in a number of indications. Cells are isolated from surgically derived tissue and are at least partially disaggregated from each other. The heterologous cell mixture is mixed with growth factors, differentiation agents, extracellular matrix proteins and/or microspheres and injected into the patient without cell expansion. The harvesting of tissue, cell isolation, and injection are performed within a single surgical procedure lasting only minutes to hours.

### CLAIM TO PRIORITY

The present application claims priority to U.S. provisional patent application No. 60/784,305, filed Mar. 21, 2006. The identified provisional application is hereby incorporated by reference.

### FIELD OF THE INVENTION

The invention relates to a non-expansion protocol for the preparation of an injectable autologous cell mixture used to prevent symptoms in a number of indications. Cells are isolated from surgically derived tissue and are at least partially disaggregated from each other. The heterologous cell mixture is mixed with growth factors, differentiation agents, extracellular matrix proteins and/or microspheres and injected into the patient without cell expansion. The harvesting of tissue, cell isolation, and injection are performed within one surgical procedure lasting only from minutes to hours.

### BACKGROUND OF THE INVENTION

### Urinary Incontinence

In the United States and Europe, urinary incontinence is believed to affect over fifty million women and over 800,000 men. More than 600,000 surgeries are performed on women and more than 10,000 surgeries are performed on men each year to address urinary incontinence. The social implications for an incontinent patient include loss of self-esteem, embarrassment, restriction of social and sexual activities, isolation, depression and, in some instances, dependence on caregivers. Incontinence is believed to be one of the most common reasons for institutionalization of the elderly.

There are five basic types of incontinence: stress incontinence, urge incontinence, mixed incontinence, overflow incontinence and functional incontinence. Stress urinary incontinence (“SUI”) is the involuntary loss of urine that occurs due to sudden increases in intra-abdominal pressure resulting from activities such as coughing, sneezing, lifting, straining, exercise and, in severe cases, even simply changing body position. Urge incontinence, also termed “hyperactive bladder” “frequency/urgency syndrome” or “irritable bladder,” occurs when an individual experiences the immediate need to urinate and loses bladder control before reaching the toilet. Urge urinary incontinence is thought to involve overactivity in the detrusor muscle (which contracts to expel urine from the bladder) and leads to a number of symptoms including urge sensation, increased urinary frequency, and nocturia. Detrusor overactivity may result from interference with normal neurological function or from defects in detrusor muscle cells that result in hypersensitivity to excitatory stimuli. Mixed incontinence is a combination of the symptoms for both stress and urge incontinence and is the most common form of urinary incontinence. Overflow incontinence is a constant dripping or leakage of urine caused by an overfilled bladder. This form of incontinence accounts for approximately 10-15% of incontinence cases and is often caused by a blockage or obstruction of the outlet from the bladder (such as from an enlarged prostate). Functional incontinence results when a person has difficulty moving from one place to another. It is generally caused by factors outside the lower urinary tract, such as deficits in physical function and/or cognitive function and accounts for about one quarter of incontinence cases.

A variety of treatment options are currently available to treat incontinence. Some of these treatment options include external devices, behavioral therapy (such as biofeedback, electrical stimulation, or Kegel exercises), injectable materials for bulking bladder sphincter or periurethral tissues, prosthetic devices to control urine flow (such as artificial sphincters) and surgery. Depending on age, medical condition, and personal preference, surgical procedures can be used to completely restore continence.

One type of procedure, found to be an especially successful treatment option for SUI in both men and women, is a sling procedure. A sling procedure is a surgical method involving the placement of a sling to stabilize or support the bladder neck or urethra. There are a variety of different sling procedures. Slings used for pubovaginal procedures differ in the type of material and anchoring methods. In some cases, the sling is placed under the bladder neck and secured via suspension sutures to a point of attachment (e.g. bone) through an abdominal and/or vaginal incision.

Another procedure, the TVT Tension-free Vaginal Tape procedure utilizes a Prolene™ nonabsorbable, polypropylene mesh. The mesh is a substantially flat, rectangular knitted article. The mesh includes a plurality of holes that are sized to allow tissue ingrowth to help avoid infection. A plastic sheath surrounds the mesh and is used to insert the mesh. During the sling procedure, incisions are made in the abdominal (i.e. suprapubic) area and in the vaginal wall. Two curved, needle-like elements are each connected to an end of the vaginal sling mesh. A sling-free end of one of the needle-like elements is initially pushed through the vaginal incision and into the periurethral space. Using a handle attached to the needle, the needle is angulated laterally (for example, to the right) to perforate the endopelvic fascia, guided through the retropubic space and passed through the abdominal incision. The handle is disconnected and the needle is then withdrawn through the abdominal wall, thereby threading a portion of the sling through the tissue of the patient. The handle is then connected to the other needle and the technique is repeated on the contralateral side, so that the mesh is looped beneath the bladder neck or urethra. The sling is positioned to provide appropriate support to the bladder neck or urethra. Typically a Mayo scissors or blunt clamp is placed between the urethra and the sling to ensure ample looseness of the sling. When the TVT mesh is properly positioned, the cross section of the mesh should be substantially flat. In this condition, the edges of the mesh do not significantly damage tissue. The sling ends are then cut at the abdominal wall, the sheath is removed and all incisions are closed. Also, an artificial sphincter may be introduced surgically to gain control over urinary emissions.

In addition to surgical procedures that alter positions of the bladder or bladder neck, bulking agents may be injected either directly into the sphincter or into spaces around the urethra. These bulking agents are believed to increase resistance to the flow of urine into and through the urethra giving the patient greater control over urinary emissions. A number of agents have been employed as periurethral bulking agents including cross-linked collagen, carbon coated beads and a biocompatible copolymer implant (e.g., Tegress™ Urethral Implant). Re-absorption by the body can limit long term effectiveness of this approach, especially for cross-linked collagen.

Autologous chondrocytes, autologous skeletal and smooth muscle, along with autologous fat are other implant materials that have been investigated. Injection of autologous fat (adipose tissue) may provide relief from symptoms of SUI, but the tissue is often resorbed by the body thereby providing only short term relief. Treatments involving injection of chondrocytes and autologous smooth muscle cell treatments are also believed to be short lived in effectiveness. Moreover, use of these cells requires biopsy and extended periods of cell culture under carefully controlled conditions to expand cell populations to the point of having enough cells to inject. Skeletal muscle cells have also been used for injection into the bladder sphincter and to periurethral regions. The approaches that have been described for use of skeletal muscle similarly require cell culture techniques to select cell subpopulations from a biopsy for injection and may require expansion of those cell subpopulations in extended culture to obtain sufficient cellular material for injection.

Thus, there is a desire to obtain a minimally invasive yet effective surgical procedure to treat incontinence, specifically stress urinary incontinence, that can be used with minimal to no side effects. Such a procedure should reduce the complexity of current procedures.

### Prolapse

Pelvic organ prolapse is defined as the decent of one or more abdominal organs (including the small bowel, uterus, bladder, rectum, urethra, and vagina) from a normal abdominal location. Prolapse involving the small bowel or uterus may lead to prolapse of the vagina, even to the point of eversion from the body. Prolapse may lead to varying degrees of discomfort in patients, to incontinence of varying severity and to other effects including painful intercourse. It is estimated that seven million women may have severe prolapse and over 600,000 surgeries are performed in the United States and Europe to address the sequellae of prolapse.

Prolapse is thought to be caused by injury to anatomic supports that normally hold the pelvic organs in place or by other dysfunction that allows the pelvic organs to descend. A number of connective tissues, including the endopelvic fascia, vesicovaginal adventitia, pubocervical fascia and rectovaginal fascia all provide support to abdominal organs. Damage to connective tissue, muscle and nerves innervating muscle attached to pelvic organs, directly or indirectly, is thought to account for a significant portion of prolapse cases. This damage may come from repeated exertion of muscles over time, such as during pregnancy, from repeated heavy lifting or even from chronic coughing. Damage to connective tissue may also come from less frequent, but more traumatic events, such as birth by vaginal delivery or hysterectomy.

Selection of surgical treatment for a prolapse condition is governed in large part by the organs affected, as well as by the severity of the condition, the involvement of other organs and potentially the existence of other medical conditions. Surgery is frequently effective in restoring the affected pelvic organs to their appropriate position. It is recognized, however, that surgical procedures to correct prolapse involving one set of organs, may lead to prolapse involving other organs.

Thus, there is a desire to obtain a minimally invasive yet effective surgical procedure to treat pelvic organ prolapse that can be used with minimal to no side effects. Such a procedure should reduce the complexity of current procedures.

### Fecal Incontinence

Fecal incontinence may result from a number of causes and many may suffer transient fecal incontinence simply as a result of loose stool or diarrhea. On the other hand, constipation can also lead to fecal incontinence when watery stool leaks around impacted stool and past anal sphincters stretched by the stool. Longer term fecal incontinence may result from pelvic floor disorders, including herniation of the rectum into the vagina or rectal prolapse. Nerve damage affecting sensory or motor control in the anal sphincter muscles may also lead to fecal incontinence. Such damage may arise during surgery or from traumatic injury. Damage to the anal sphincter muscles themselves can lead to loss of control over the contraction of the sphincters, leading to incontinence. One of the major causes of damage to anal sphincter muscle results from vaginal delivery of children. More than five million people in the United States are believed to be affected by fecal incontinence.

Treatments for fecal incontinence include diet changes (including addition of fiber to the diet) and bowel training systems to achieve regularity. Medications, such as antidiarrheal medications can give patients more control over bowel movements by controlling rectal contractions or by providing additional consistency to the stool. A number of different surgeries may be used to address fecal incontinence, depending on the cause and severity of the problem. In sphincteroplasty, a sphincter is cut in the region of a defect or injury and the two ends are overlapped and then sewn in place. In other cases, muscle transposition is used to repair the sphincter by surrounding the anal canal with skeletal muscle (from forearm, thigh or buttock) to allow for restoration of voluntary control. Artificial sphincters may also be used to provide assurance of control over passing of stool. Protocols for injection of bulking agents into the anal sphincter or the regions surrounding the anal sphincter are receiving increased attention; however the use of these agents is still limited.

Thus, there is a desire to obtain a minimally invasive yet effective surgical procedure to treat fecal incontinence that can be used with minimal to no side effects. Such a procedure should reduce the complexity of current procedures.

### Erectile Dysfunction

Erectile dysfunction is believed to affect more than ninety million men in the United States and Europe, with seventeen million presenting with severe conditions that greatly interfere with the ability to initiate and maintain erections. Erectile dysfunction may arise from a number of causes. Age brings on a lack of arterial elasticity in vessels supplying blood to erectile tissues. Damage to nerves necessary for initiating and sustaining erections brought on by chronic conditions (such as diabetes) or by injury can lead to dysfunction. A significant cause of nerve damage comes from injury that occurs during prostate surgeries, especially radical prostatectomies. Although new surgical procedures have been introduced that conserve the nerves in this region, a majority of men who undergo can still expect some degree of post operative erectile dysfunction.

A number of oral medications for treating erectile dysfunction have entered the marketplace in recent years, including VIAGRA, CIALIS and LEVITRA. These medications all provide significant relief to a large segment of men with erectile dysfunction. However, they each require that the medication be taken in advance of initiation of sexual activity and their effects may be delayed if ingested with food.

Various treatments have also been tried in connection with erectile dysfunction, including administration of Prostaglandin E1 by injection into the cavemosum of the penis, by administration of a suppository into the urethra and by topical administration. These approaches allow for less advance preparation, but are not highly effective across patient populations, especially radical prostatectomy patients.

Surgical interventions are also available for addressing erectile dysfunction, especially where medications are ineffective or contraindicated. Penile implants of many different configurations are used to provide support for an erection. These implants are effective in restoring patient sexual satisfaction. Increasingly, these implants have been engineered to be completely concealed within the patient. However, implants may fail over time and replacement or total removal may be required potentially leaving the patient with no relief at all.

Thus, there is a desire to obtain a minimally invasive yet effective surgical procedure to treat erectile dysfunction that can be used with minimal to no side effects. Such a procedure should reduce the complexity of current procedures.

### Interstitial Cystitis

Interstitial cystitis is a progressive syndrome affecting the urinary bladder and may present in ulcerative (or classic) or nonulcerative forms. Symptoms associated with interstitial cystitis include increased urgency and frequency of urination, as well as pelvic pain. Patients afflicted with interstitial cystitis also complain of more generalized symptoms that affect quality of life, often significantly, including chronic abdominal pain. Origin of this syndrome in patients is not well understood. While evidence of increased immune function in the region of bladder muscle has been observed in patients (typically higher numbers of immune system cells), no bacterial or other agents have been consistently associated with this syndrome.

Numerous oral agents have been tested for treatment of interstitial cystitis. These agents include L-arginine, pentosan polysodium sodium, cimetidine, gabapentin, suplatast tosilate (an immunoregulator), quercetin, Nerve Growth Factor (NGF) and montelukast (a leukotriene receptor antagonist). Intravesical treatments have also been evaluated. Lidocaine, heparin, BCG, hyaluronic acid and vanilloids have shown varying degrees of success in relieving symptoms. Interventional treatments, such as sacral neuromodulation have been tried, but these are costly in the long term and invasive. Surgical treatment for interstitial cystitis may involve ablation procedures or in severe cases, removal of the bladder. This radical approach is very often successful in alleviating symptoms if interstitial cystitis. However, patients will likely desire a bladder substitute to maintain as normal a lifestyle as possible, thereby requiring additional surgery.

Thus, there is a desire to obtain a minimally invasive yet effective surgical procedure to treat interstitial cystitis, and that can be used with minimal to no side effects. Such a procedure should reduce the complexity of current procedures.

### SUMMARY OF THE INVENTION

Cell-based therapies using autologous stem cells require long expansion periods which can add weeks to the procedure, necessitating additional personnel and equipment. In addition, increased culture time amplifies contamination risk, selects for specific cell populations within any given cell type, and has been associated with cellular changes. This invention, and various other related inventions disclosed herein, eliminates, or substantially eliminates, the expansion step by retaining all or a portion of mixed cells in the surgically derived specimen rather than selecting for a specific subpopulation that must be expanded to achieve sufficient cell numbers for injection. In order to obtain an injectable (or similar means for re-introduction, not necessarily limited to injection) solution of heterologous cells, the tissue is disaggregated using mechanical and enzymatic means or processes. The cells are collected and resuspended in a physiological media that may contain growth factors, differentiation agents, extracellular matrix proteins, and/or microspheres. The cellular solution may then be injected at the site of tissue defect or tissue (or muscle) degeneration. This method reduces the time from tissue removal to injection (or re-introduction) from weeks to hours (and possibly less, depending on the enzymatic processes or other means to speed up the disaggregation process) and therefore, requires only one hospital or office visit.

### BRIEF DESCRIPTION OF THE DRAWINGS

FIG. 1 is a flow chart detailing an embodiment of the method of the present invention.

### DETAILED DESCRIPTION OF THE PREFERRED EMBODIMENTS

The present invention provides for a system and method for obtaining a solution of heterologous cells for introduction to the body. According to a method of the present invention, tissue is disaggregated using mechanical and enzymatic means or processes. Cells are collected and resuspended in a physiological media that may contain growth factors, differentiation agents, extracellular matrix proteins, and/or microspheres. The cellular solution may then be injected at the site of tissue defect or tissue (or muscle) degeneration. Notably, the method of the present invention requires no culturing and requires no isolating of a specific cell population.

### EXAMPLE 1

### Nonexpansion Protocol for Isolation of Autologous Cells for Cell-Based Therapy

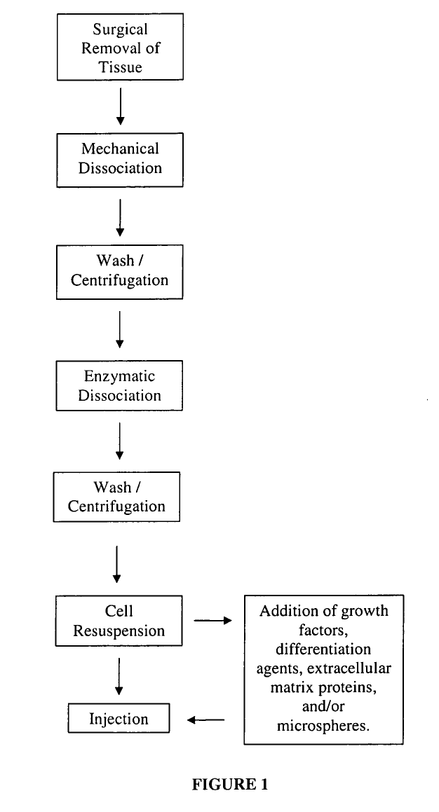

In this example embodiment, cells are harvested from skeletal muscle biopsies or otherwise surgically derived tissue by mechanical and enzymatic means. See FIG. 1 as one example of a flowchart that helps to describe the method of this and other embodiments of the present invention. The tissue is first finely chopped on ice (or other cold and sterile surface) using a sterile scalpel and then washed three times in a sterile balanced salt solution optionally containing antibiotics and/or antimycotics. The cells are enzymatically dissociated (or via another dissociating means or process) by incubating with cell dissociation enzymes (e.g. any of trypsin, collagenase, or dispase and combinations thereof) for about one hour (or until the cells are sufficiently dissociated) at about 37° C. This embodiment is not necessarily limited to this time and temperature. After the enzymatic dissociation, the cells are washed about three times by centrifugation in a sterile balanced salt solution and resuspended at an effective concentration that may be in the range of about 10⁵-10⁷ cells/ml in a sterile isotonic solution. Cellular solutions may contain various combinations of multiple cells types. Possible cell types include but are not necessarily limited to myoblasts, fibroblasts, nerve cells, endothelial cells, and adipocytes. In addition to cells, growth factors or differentiation agents and a biocompatible carrier that could be an extracellular matrix component or microspheres, may be added to the injectable cellular solutions. Advantages of injectable non-cellular components include: improved cell survivability, cell differentiation, cell retention, cell grafting, injection visualization, and providing temporal bulking effect.

### EXAMPLE 2

### Determination of In Vivo Retention Time, Survival, and Localization

In this example embodiment, a method is described for determining retention time, survival and localization of cells injected (or re-introduced) into the bladder.

Cell Preparation and Transplantation

Cells are isolated from muscle taken from the hind limb of newborn normal mice (or similar mammal) using mechanical and enzymatic means and then plated on 60 mm gelatin-coated plates in DMEM containing 10% fetal bovine serum, 10% horse serum, 1% glutamine (292 μg/ml), penicillin (100 U/ml) and streptomycin (100 μg/ml) (all from Invitrogen). The cells are incubated at about 37° C. for about 2 hours and then fluorescent latex microspheres (FLMs) are added at a dilution of 1:3000 to the cultures. After about 12 hour incubation at about 37° C., the cells are rinsed about three times with HBSS, detached with 0.25% trypsin-EDTA, and resuspended in HBSS at a concentration of 10⁶ cells/ml. Ten microliters of the heterologous cellular suspension is injected into both the left and right sides of the detrusor muscle in adult mice.

Follow Up Testing

Fourteen, twenty eight, and fifty six days after cell transplantation, the mice are sacrificed and the bladder is dissected out. The muscle is rinsed in phosphate buffered saline (PBS), incubated in 30% sucrose in PBS for 12 hours, and frozen. The frozen specimens are sectioned using a cryostat, mounted on gelatin-coated slides and air-dried. After drying, the slides are rinsed in PBS. The cover slip is mounted using Fluoromount (Atomergic Chemetals Corp.) and sealed using clear nail polish. Immunohistochemistry for dystrophin and detection of the FLMs with a fluorescent microscope are used to determine the survival of the injected cells and their presence in myofibers.

### EXAMPLE 3

### Treatment of Stress Urinary Incontinence in Rats Using Cell-Based Therapy

This Example embodiment describes a treatment of stress urinary incontinence (SUI) in a rat model by injecting a mixture of nonexpanded, heterologous cells. This particular protocol may be useful in treating women having hysterectomies that also have stress urinary incontinence or in men exhibiting stress incontinence after a prostatectomy.

Animals

Female Sprague-Dawley rats, age 3-6 months, are used as a source of cells and as treatment animals. The animals are handled in compliance with the requirements of the Institutional Animal Care and Use Committee (IACUC) at our animal facility.

Description of Animal Model

The procedure is performed on female rats with electrocauterization-induced intrinsic sphincter deficiency (1) [Chemansky et al. “A model of intrinsic sphincteric deficiency in the rat: electrocauterization.” Neurolurol Urodyn. 2004; 23(2): 166-71]. This model decreases leak point pressure (LPP) without affecting the bladder. LPP measures continence by analyzing the abdominal pressure required to cause urethral leakage. The LPP of cauterized rats is compared to that of animals that underwent a sham operation at day 0.

Cell Harvesting

a) The cells are isolated from a surgically extracted uterus taken from a normal female adult Sprague-Dawley rat. Upon extraction of the uterus, the peritoneum and mucous membranes are removed and the remaining muscular layer is placed in ice cold Hank's Balanced Salt Solution (HBSS) without calcium or magnesium (Invitrogen) and containing 100 units/ml penicillin, 100 μg/ml streptomycin, and 0.25 μg/ml amphotericin B (Invitrogen). Upon gross examination, any abnormalities are removed. Various cell types may be isolated including myoblasts, satellite cells, muscle stem cells, fibroblasts, endothelial cells, and nerve cells.

b) Cell Disaggregation. The muscle is finely chopped into about 3-4 mm pieces on ice using a sterile scalpel and then washed about three times in HBSS containing penicillin, streptomycin, and amphotericin B at the previously mentioned concentrations. The cells are enzymatically dissociated by incubating with about 0.25% trypsin-EDTA (Invitrogen), 0.2% collagenase (Sigma) in HBSS for about one hour at about 37° C. After the enzymatic dissociation, the cells are washed three times by centrifugation in HBSS (300 rpm for 5 minutes at room temperature) and resuspended at 10⁶-10⁷/ml in a sterile isotonic solution such as HBSS or phosphate buffered saline. The solution may also contain extracellular matrix components such as glutaraldehyde cross-linked bovine collagen or microspheres and growth factors including but not limited to 50 ng/ml vascular endothelial growth factor and nerve growth factor (both from Invitrogen) to improve muscle healing and function.

Injection

The animals are anesthetized and after surgical preparation, a midline incision is made to expose the proximal urethra. The cellular solution is injected into multiple sites of the proximal urethral submucosa of both cauterized and sham-operated female Sprague-Dawley rats 3-6 months of age. An equal number of cauterized and sham-operated female rats are also injected with an equal volume of sterile isotonic solution. Using a Hamilton micro syringe, 10 μl is injected per site. Each animal has the same number of injections ranging from 2-4 in the same relative locations.

Follow Up Testing

Sphincter function is assessed by measuring LPP. The LPPs of all four groups (cauterized, treated; cauterized control; sham, treated; sham, control) are compared at 4, 7, 14, and 28 days.

### EXAMPLE 4

### Treatment of Erectile Dysfunction in Rats Using Cell Based Therapy

This Example embodiment describes the treatment of post-radical prostatectomy erectile dysfunction (ED) with a heterologous cellular mixture including but not limited to myoblasts, endothelial cells, and nerve cells isolated from the hind limb of a normal adult Sprague Dawley rats.

Post-Radical Prostatectomy Erectile Dysfunction Model

Bilateral transection of the cavernous nerves in rats is used as a model of post-radical prostatectomy erectile dysfunction. The rats are randomly divided into three experimental groups that include a control group, a nerve transected group injected with vehicle, and a nerve transected group injected with a cellular mixture that may include growth factors.

Cell Preparation and Transplantation

Cells are isolated from the hind limbs by first dissecting away the skin and bones, and chopping the muscle into a coarse slurry using sharp scalpels. The muscle slurry is then washed with HBSS containing antibiotics and an antimycotic and incubated at about 37° C. with a series of enzymes including 0.2% collagenase for about 45 minutes, 2.4 U/ml dispase (Invitrogen) for about 30 minutes, and 0.25% trypsin for about 15 minutes. After the enzymatic dissociation, the cells are washed about three times by centrifugation in HBSS (about 300 rpm for about 5 minutes at room temperature) and resuspended at 10⁵-10⁶ cells/ml in a sterile isotonic solution containing about 50 ng/ml of each of the following growth factors: vascular endothelial growth factor (VEGF), basic fibroblast growth factor (bFGF), and brain-derived neurotrophic factor (BDNF) (all from Invitrogen). Ten microliters of the vehicle or the cellular mixture are injected at the site of injury. At various time points after injection, intracavernousal pressure (ICP) of all experimental groups is measured during electrostimulation of the pelvic nerve.

### EXAMPLE 5

### Treatment of Tissue Defects and Degeneration Using Cell Based Therapy

The present invention may also be used to treat human urological and gynecological disorders involving tissue defects and degeneration. These disorders include but are not limited to stress urinary incontinence, prolapse, fecal incontinence, erectile dysfunction, and interstitial cystitis. An effective amount of a nonexpanded, autologous cell mixture that may contain growth factors can be injected at the site of defect so that function is restored to the tissue. The injection site will vary depending on the condition. Stress urinary incontinence often results from damage to the urinary sphincter or to the connective tissue supporting the urethra and bladder neck. Injection into these areas could restore continence. Prolapse could be treated by injecting a cellular mixture into weakened or damaged connective tissue whose normal function is to support pelvic structures. Fecal incontinence is commonly caused by nerve or muscle damage to the anus and could be reduced by injecting a cellular mixture containing nerve cell growth factors into the weakened or damaged internal and external sphincters. Erectile dysfunction results from nerve damage or reduced blood flow in the penis. Injection of a cellular mixture containing growth factors that promote nerve and/or endothelial cell growth into the corpus cavemosa could act to repair or replace damaged nerves and/or endothelial cells and enhance function. For interstitial cystitis, a cellular mixture could be injected into the bladder in order to repair damaged tissue.

The above examples each describe an application of the non-expansion protocol for the preparation of an injectable autologous cell mixture of the present invention that can be used to prevent symptoms in a number of indications. Cells are isolated from surgically derived tissue and are at least partially disaggregated from each other. The heterologous cell mixture is mixed with growth factors, differentiation agents, extracellular matrix proteins and/or microspheres and injected into the patient without cell expansion. The harvesting of tissue, cell isolation, and injection are performed within a single surgical procedure lasting only from minutes to hours.

While this invention has been described with an emphasis upon preferred embodiments, it will be obvious to those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

What is claimed:

1. A method for treating a pelvic tissue anomaly in a patient, comprising:
obtaining a portion of tissue from a patient isolating cells within said tissue;
disaggregating at least a portion of said cells within said tissue, wherein said steps of isolating and disaggregating produce a heterologous cell mixture;
combining said heterologous cell mixture with a mixing agent to produce an injectable solution;
injecting said injectable solution into said patient proximate the pelvic tissue anomaly, wherein each of said steps is performed during a single medical procedure and wherein said single medical procedure is performed within a time period of one minute to five hours.

2. The method according to claim 1, wherein the said method is performed without the steps of culturing and isolating a specific cell population.

3. A method according to claim 1, wherein the cells are autologous.

4. A method according to claim 1, wherein the cells are not passaged.

5. The method of claim 1, wherein said mixing agent is selected from a group comprising: a physiologically acceptable medium, a growth factor, a differentiation agent, an extracellular matrix protein, microspheres or other non-cellular agents that would provide an initial bulking effect and/or improve cell survival, or a combination of one or more of the mixing agents.

6. The method according to claim 4, wherein the growth factor is a member of a growth factor family selected from a group consisting of the VEGF family, the FGF family, the EGF family, the IGF family, and the TGF-beta family.

7. The method according to claim 4, wherein the growth factor is selected from the group consisting of basis fibroblast growth factor (b-FGF), insulin-like growth factor-1 (IGF-1), nerve growth factor (NGF), and brain-derived neurotrophic factor (BDNF).

8. The method according to claim 4, wherein the extracellular matrix protein is selected from the group consisting of collagen, fibronectin, laminin, vitronectin, heparin, heparan sulfate, chondroitin sulfate, dermatan sulfate, and hyaluronate.

9. The method according to claim 4, wherein the microspheres or bulking agents are selected from a group consisting of poly(lactic-co-glycolic acid), polylactic acid, polyglycolic acid, poly(orthoesters), polyanhydrides, polycaprolactone, polyhydroxybutyrate, polyethylene terephthalate, polyarylates, polylactic acid-polyethylene glycol copolymer, poly(lactic-co-glycolic acid)-polyethylene oxide copolymer, polydioxanone, poly-trimethylene carbonate, polyester amides, polyglycolic acid-co-trimethylene carbonate, polyhydroxy butyrate valerate, polyphophagenes, polyurethane, silicone, carbon biospheres, polystyrene, polyvinyl acetate, chitosan, alginate, albumin, and polyamino acids.

10. The method of claim 1, wherein said pelvic tissue anomaly for treatment is selected from a group comprising:
urinary incontinence (stress, urge, mixed, neurogenic, overflow);
fecal incontinence;
prolapse;
erectile dysfunction;
interstitial cystitis;
urinary retention disorder;
female sexual dysfunction;
male sexual dysfunction;
pelvic pain;
pelvic pain and organic dysfunction;
prostatalgia;
cancer; and
abnormal uterine bleeding.
